(19) United States
(12) Patent Application Publication
(10) Pub. No.: US 2016/0022858 A1
(43) Pub. Date: Jan. 28, 2016

(54) AIR FRESHENER WITH CLIP

(71) Applicant: Scent2Market Inc., Yonkers, NY (US)

(72) Inventor: Daniel M. D'Amico, South Salem, NY (US)

(73) Assignee: Scent2Market Inc., Yonkers, NY (US)

(21) Appl. No.: 14/874,846

(22) Filed: Oct. 5, 2015

Related U.S. Application Data
(63) Continuation-in-part of application No. PCT/US2014/044130, filed on Jun. 25, 2014.
(60) Provisional application No. 61/839,138, filed on Jun. 25, 2013.

(51) Int. Cl. A61L 9/04; A01M 29/12; A61L 9/12

(52) U.S. Cl. CPC A61L 9/042; A61L 9/048; A61L 9/12; A01M 29/12

(57) ABSTRACT

The disclosure provides a fragrance distribution device including a housing. The housing has an aperture into which a hook, clip, adhesive peg, or other attaching device may be secured. This allows the housing to be placed into a vent or clipped to a surface at the convenience of a user. Typically the housing encloses a thermoplastic fragrance carrier. Other embodiments include a housing having hingedly connected compartments to selectively envelop and retain a carrier/support within cavity portions of the compartments.

### CROSS-REFERENCE TO RELATED APPLICATIONS

This application is a continuation-in-part application of international application PCT/US2014/044130 filed on Jun. 25, 2014 and claiming priority to U.S. Provisional Application No. 61/839,138, filed on Jun. 25, 2013, both of which are incorporated by reference in their entirety.

### BACKGROUND OF THE INVENTION

1. Field of the Invention

The invention relates to the fields of scent and aroma management. This may include, for example, but not be limited to increasing the amount of desirable aroma, fragrance, or odor neutralizer in a room, a vehicle, or another area.

2. Background of the Related Art

Many devices for providing a fragrance to an area over a period of time are known. Generally, these devices operate by allowing a fragrance contained in the device to passively diffuse from the device and into the atmosphere. In some cases these devices are suspended from a loop, wire, string, or other connector, allowing them to depend from a knob, minor, or the like.

In some cases distribution of a fragrance is assisted by actively forcing air through the housing of a device. This may be done by including a fan or other air-moving device as an integral part of the fragrance device. It may also be accomplished by securing the fragrance device in close proximity to another source of moving air. One variety of the latter kind of device is shown, for example, in U.S. Pat. No. 8,460,609, which shows a fragrance device in which an automobile vent is used to provide an air flow to assist in the distribution of a fragrance. That patent is incorporated by reference herein.

Unfortunately, known solutions for fragrance distribution may suffer from a number of disadvantages. For example, a consumer may purchase a device meant to depend from a loop, but then decide that the more aggressive fragrancing made available by a secured model is either permanently or temporarily desired. In another situation a consumer using a secured fragrance device may decide that while both the air flow and the fragrance are still desired, the enhanced fragrancing provided by securing the fragrance device to a vent is not still desired. Unfortunately, such a useful device has not previously been proposed.

### BRIEF SUMMARY OF THE INVENTION

It would be helpful to have a fragrance distribution device that retained maximum flexibility in potential location of the device and fragrance delivery. Embodiments of the invention provide a fragrance distribution device comprising a housing, and a fragrance in a polymer or thermoplastic carrier. The housing includes an aperture into which a hook, clip, adhesive peg, or other attaching device may be secured. Typically the aperture is covered by a decal (also referred to as a label) or other cover until its use is desired. In some cases the attaching device is held by a recess in the air freshener when not in use.

In some embodiments the attaching device may be removably secured to the housing. By “removably secured” it is meant that the attaching device may be repeatedly removed from and replaced in the housing without damaging the housing or the attaching device. This may be accomplished, for example, by an interference fit or a screw.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 shows a fragrance device according to one embodiment of the invention, in which a housing 1 includes a label 3. An attaching device, in this case a clip 5 is also shown.

FIG. 2 shows the fragrance device of FIG. 1 with the label 3 removed from the housing 1. Through the walls of the housing (which is transparent in this embodiment but need not be transparent in all embodiments), the polymer fragrance carrier 7 is visible. A number of circular fragrance vents 9 are shown, and on each side of the housing are additional fragrance vents 11. Aperture 13 is also included. Also shown is loop 15, which is secured through the housing by hole 17.

FIG. 3 shows an assembled fragrance device including the housing 1 connected to the clip 5.

FIG. 4 and FIG. 5 show embodiments of the invention with an adhesive peg 23 and a hook 25, respectively, for attachment of the housing to an external environment.

FIG. 6 shows an embodiment in which a clip is held by a screw 27. This may be compared to the interference fit shown in FIG. 3, in which a clip is inserted and then twisted 90 degrees to be secured.

FIG. 7 shows an embodiment of the invention in which holes for fragrance are covered by a hard cover 19 movable by hinges 21 to allow the user to vary the flow of fragrance.

FIG. 8 shows an embodiment in which a clip is held by a ball and socket attachment. This figure also shows a recess 30 into which the clip may be placed when not in use.

FIG. 9 shows a fragrance device according to one embodiment of the invention, in which a housing 1 includes a label 3. An attaching device, in this case a clip 5 is also shown. The clip is within a recess, and the recess is, in turn, covered by a label.

FIGS. 10A and 10B show exterior and interior views, respectively, of a fragrance device according to one embodiment of the invention, in which the housing 1 includes a first compartment 102 hingedly attached to a second compartment 104.

FIGS. 11A-11C show a rear view, a front view, and a side view, respectively, of a fragrance device with a housing 1 having a first compartment 102 hingedly attached to a second compartment 104.

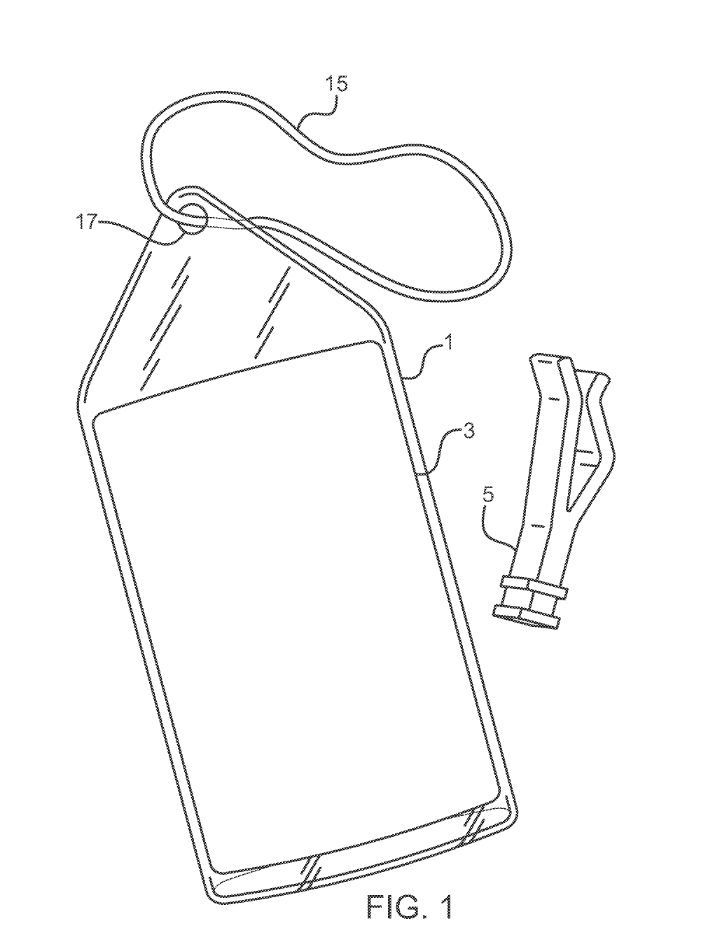

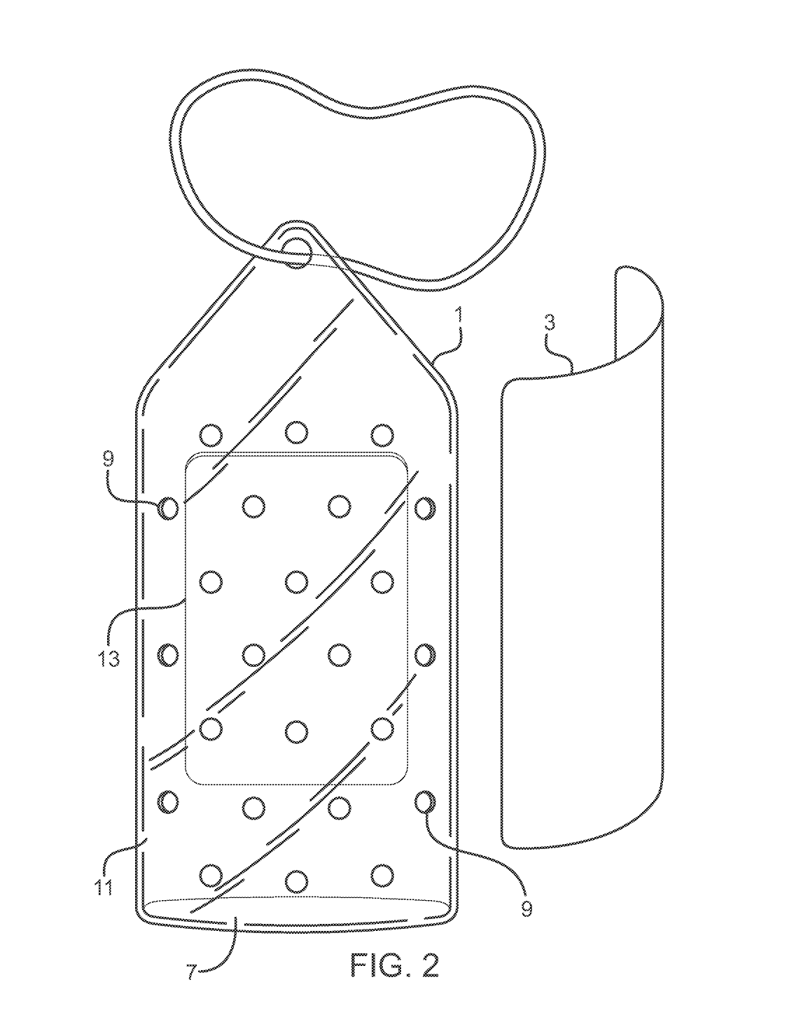

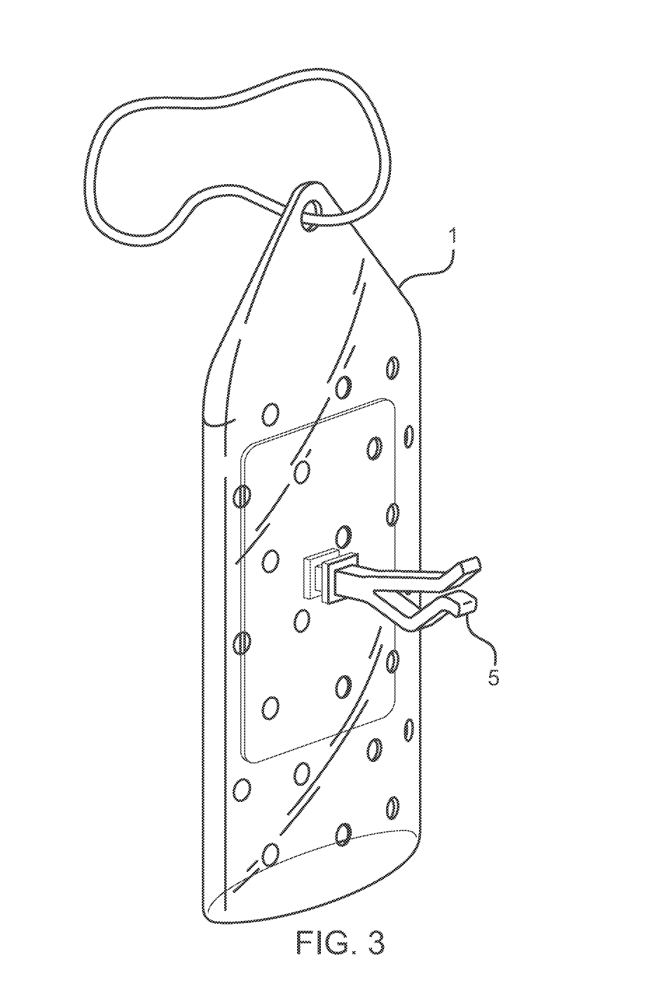

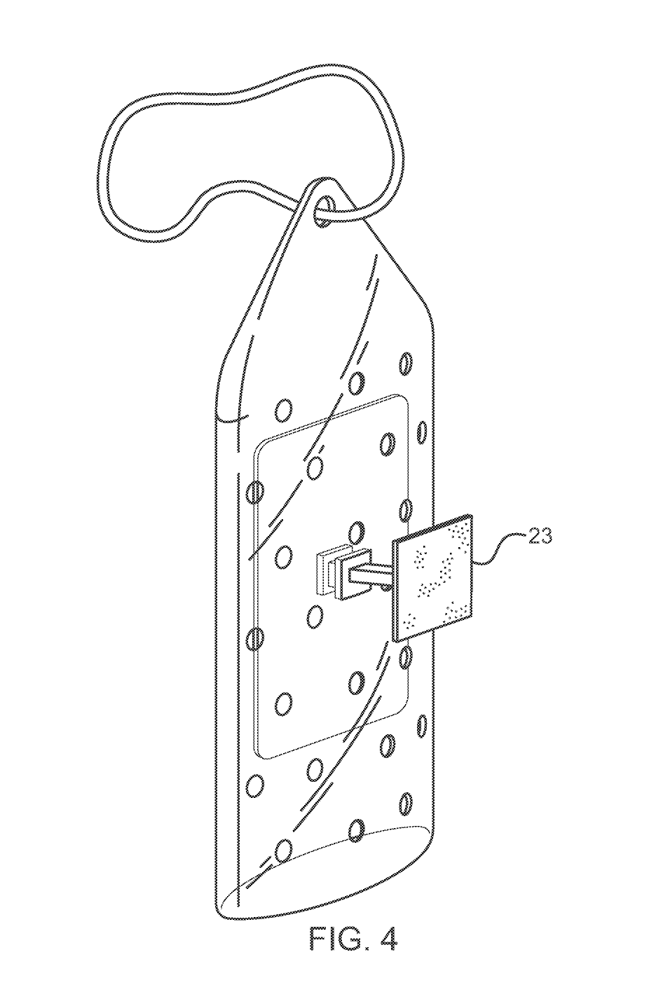

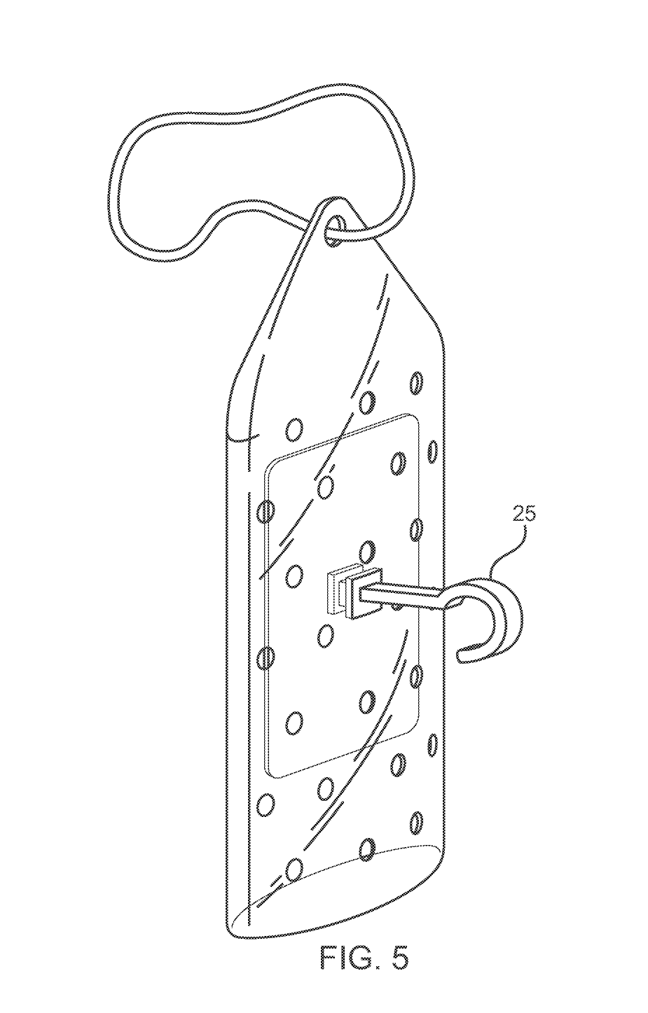

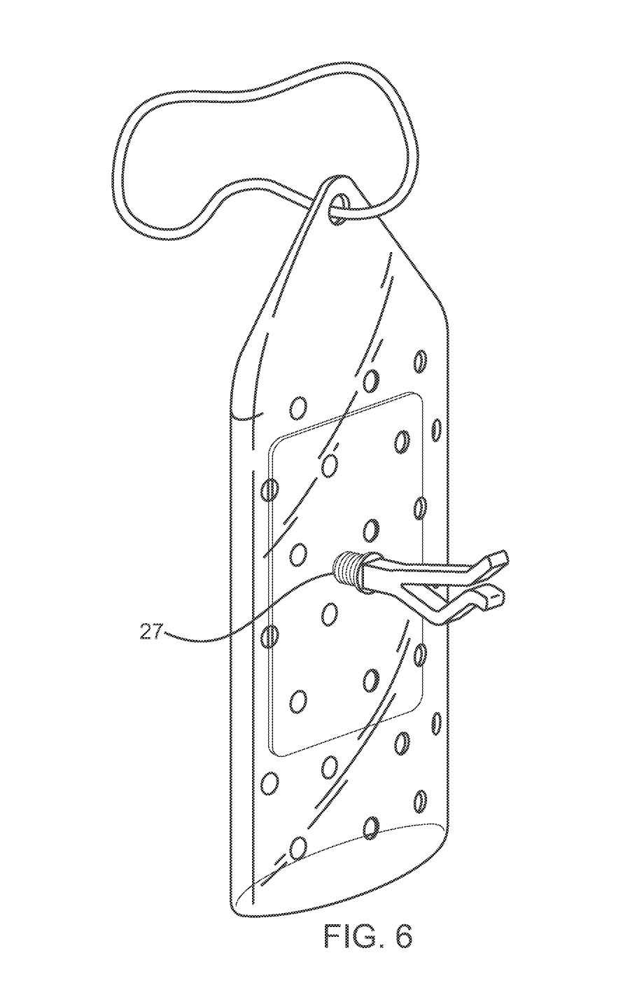

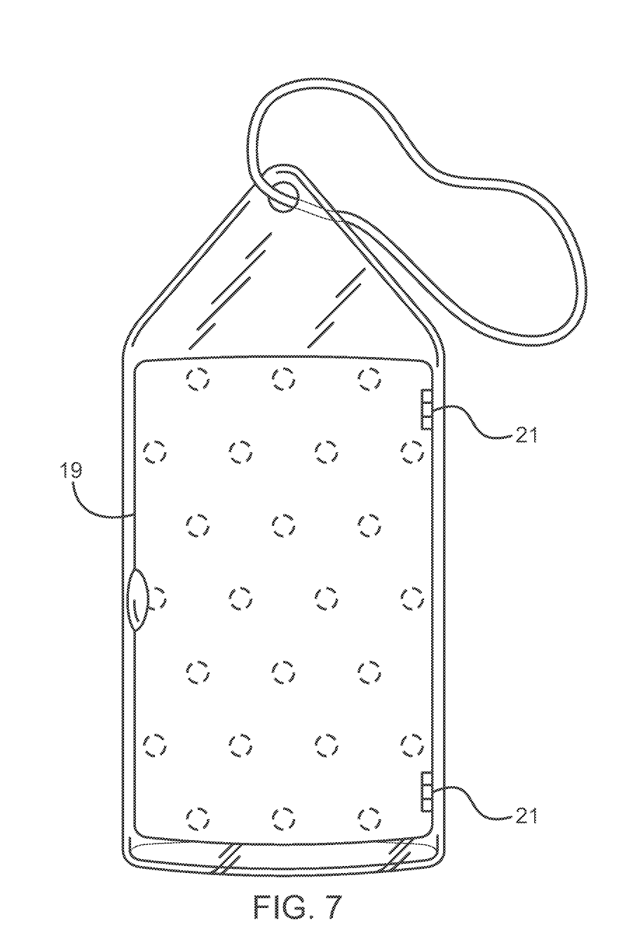

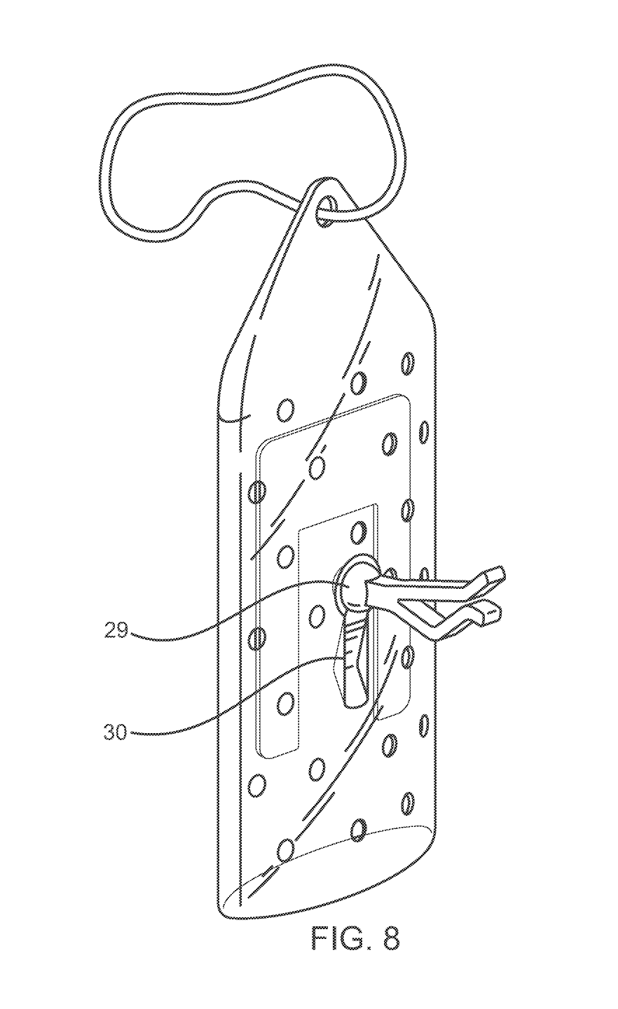

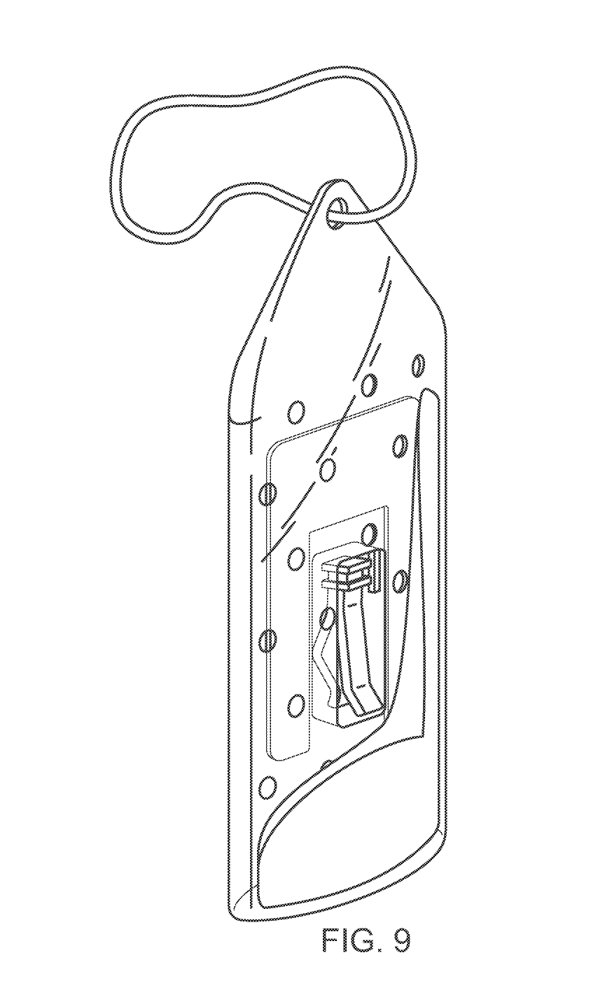

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

Embodiments of the invention provide a fragrance distribution device comprising a housing. The housing includes an aperture into which a hook, clip, adhesive peg, or other attaching device may be secured. Typically the aperture is covered by a label, decal, or other cover until its use is desired. The housing encloses a polymer or thermoplastic fragrance carrier. Typically the housing is plastic, though it may be other materials as desired, and the material of the housing is not critical.

In some embodiments the attaching device may be removably secured to the housing. By “removably secured” it is meant that the attaching device may be repeatedly removed from and replaced in the housing without damaging the housing or the attaching device. This may be done, for example, by having the attaching device able to be freely inserted into a slot, and then twisted to be secured. It may also be done by having the attaching device screw into the housing.

In typical embodiments the housing is attached to a loop or other device that will allow the device to be placed over a knob, mirror, or other protrusion. This kind of temporary placement allows the device to passively release fragrance without attachment to a vent or fan.

The aperture(s) in the fragrance device may have any of a number of shapes and configurations. For example, it may be a horizontal, vertical, or diagonal slot. It may be a circular hole. The hole may be threaded or unthreaded. The aperture may be centered in the housing or off-center.

The housing may be any shape. Typically the housing includes vents on one or more of the side, front, or reverse of the housing, allowing the fragrance to escape the housing. The vents may be any shape but are typically holes or slots. In some embodiments the vents are on the same surface as the aperture.

In some embodiments the aperture is covered when not in use. This may be done by a decal, sticker, or other adhesive. In other embodiments the aperture may be closed by a more substantial cover, for example a hard plastic cover. This cover may be attached, for example by hinges or by an interference fit. The cover may be able to be replaced after it has been removed, or its removal may be permanent. In some embodiments the covering over the aperture is punctured rather than removed.

Removal of the cover may reveal one or more vent openings in the surface of the housing. In some embodiments a user may elect to reveal less than all of the vent openings when removing the cover.

### Polymer Carriers

Embodiments of the invention include the fragrances in a polymer or thermoplastic carrier/support. One preferred polymeric fragrance carrier/support is ethylene vinyl acetate (EVA). EVA is a copolymer of ethylene and vinyl acetate. The EVA has no odor by its nature, however, it can adsorb or otherwise be permeated with a fragrance. EVA approaches elastomeric materials in softness and flexibility, yet can be processed like thermoplastics.

EVA used in the invention may have a molecular weight in the range of, for example, 10,000 Daltons to 100,000 Daltons, more preferably 22,000 to 87,000 Daltons. Fragrance may be introduced into the polymer at weight percents varying from 10 to 90%, from 20 to 80% from 30 to 70%, from 30 to 60%, and from 30 to 50%. In further embodiments, fragrance is introduced into the polymer at a weight percent of about 1%, about 10%, about 20%, about 30%, about 40%, about 50%, about 60%, about 70%, about 80%, about 90%, or about 95%.

Other elastomeric or thermoplastic carriers or combinations thereof may be used as fragrance carriers/supports, so long as they will release fragrance upon heating. Other suitable polymeric materials share the beneficial properties of EVA and may be substituted for use in embodiments of the invention. These include, for example, but are not limited to, high density polyethylene, low density polyethylene, polystyrene, acrylic polymers, polycarbonates, polyurethanes, nylons, and mixtures and copolymers of the foregoing.

Organic and/or inorganic gellants may also be used as a fragrance carrier/support. This may include odor adsorbing or fragrance impregnated gels or fragrance gallants. For example, an IFO gel (for example, a polyamide gel) is one suitable example of a gellant that may be used in embodiments of the invention to replace all or part of the polymer component. Examples of gels and gallants may include, but are not limited to, silica, carrageenan, and/or polyamide, which may be combined with other materials to enhance stiffness. Other examples of gels may be a thermoset polymer including a fragrance and epoxy so as to allow it to polymerize into a rubber-like article.

Non-polymer fragrance carriers/supports may be used as well. These may include, but are not limited to, porous ceramic, wood, pressed cellulose (blotter), etc. For example, silica or sintered aluminum oxide may be formed with sufficient porosity to enable permeation of fragrance within the article comprising the non-polymer fragrance carrier/support.

### Fragrances

One or more fragrances or odor neutralizers may be used in embodiments of the invention. If addition of fragrance is desired, suitable fragrances may be selected from those compiled by the U.S. Food and Drug Administration in Title 21 of the Code of Federal Regulations, Sections 172.510 and 172.515, incorporated by reference herein. Fragrance components selected from benzaldehydes, phenols, cinnamic aldehydes and esters, octadienes, dienes, cyclohexadienes, and terpenes may be used in the invention. Fragrance oils are also suitable for use alone or in combination with other fragrance chemicals. Suitable fragrance oils are, for example spice oil, flower oil, and fruit oil. Although fragrances are featured in many embodiments as reported here, other things deliverable by air including odor neutralizers and insect repellents may also be used.

Other suitable fragrances include but are not limited to benzyl alcohol, ethyl maltol, furaneol, 1-hexanol, cis-3-hexen-1-ol, menthol, benzaldehyde, hexanal, cinnamaldehyde, citral, cis-3-hexenal, furfural, neral, vanillin, ethyl acetate, ethyl butanoate, ethyl decanoate, ethyl hexanoate, ethyl octanoate, hexyl acetate, isoamyl acetate, methyl butanoate, methyl salicylate, pentyl butanoate, pentyl pentanoate, sotolon, strawberry aldehyde, fructone, anethole, anisole, eugenol, dihydrojasmone, 2-acetyl-1-pyrroline, 6-acetyl-2,3,4,5-tetrahydropyridine, gamma-decalactone, gamma-nonalactone, delta-octalactone, jasmine lactone, massoia lactone, camphor, citronellol, linalool, nerol, nerolidol,alpha-terpineol, thujone, and thymol.

In further embodiments of the invention, fragrances and/or odor neutralizers are mixed with one or more hindered amines. The hindered amines useful in the instant invention are well known in the art and are described in detail in U.S. Pat. No. 6,221,115, the relevant parts of which are incorporated herein by reference. Examples of the hindered amines are: 1-(2-hydroxy-2-methylpropoxy)-4-octadecanoyloxy-2,2,6,6-tetramethylpiperi-dine; 1-(2-hydroxy-2 -methylpropoxy)-4-hydroxy-2,2,6,6-tetramethylpiperidin-e; bis(1-octyloxy-2,2,6,6-tetramethylpiperidin-4-yl) sebacate; bis(1-cyclohexyloxy-2,2,6,6-tetramethylpiperidin-4-yl) sebacate; 1-cyclohexyloxy-2,2,6,6-tetramethyl-4-octadecylaminopiperidine; 2,4-bis [(1-cyclohexyloxy-2,2,6,6-tetramethylpiperidin-4-yl)butylamino]-6-(2-hydroxyethylamino-s-triazine; bis(1-cyclohexyloxy-2,2,6,6-tetramethylpiperidin-4-yl) adipate; 1-(2-hydroxy-2-methylpropoxy)-4-oxo-2,2,6,6-tetramethylpiperidine; bis(1-(2-hydroxy-2-methylpropoxy)-2,2,6,6-tetramethylpiperidin-4-yl) sebacate; bis(1-(2-hydroxy-2-methylpropoxy)-2,2,6,6-tetramethylpiperidin-4-yl) adipate; bis(1-(2-hydroxy-2-methylpropoxy)-2,2,6,6-tetramethylpiperidin-4-yl) succinate; bis(1-(2-hydroxy-2methylpropoxy)-2,2,6,6-tetramethylpiperidin-4-yl) glutarate; and 2,4-bis{1N-[1-(2-hydroxy-2-methylpropoxy)-2,2,6,6-tetramethylpiperidin-4-y-1]-N-butylamino}-6-(2-hydroxyethylamino)-s-triazine) 1-methoxy-4-hydroxy-2,2,6,6-tetramethylpiperidine; 1-methoxy-4-hydroxy-2,2,6,6-tetramethylpiperidine; 1-octyloxy-4-hydroxy-2,2,6,6-tetramethylpiperidine; 1-cyclohexyloxy-4-hydroxy-2,2,6,6-tetramethylpiperidine; 1-methoxy-4-oxo-2,2,6,6-tetramethylpiperidine; 1-octyloxy-4-oxo-2,2,6,6-tetramethylpiperidine; and 1-cyclohexyloxy-4-oxo-2,2,6,6-tetramethylpiperidine, or a mixture thereof.

In yet further embodiments of the invention, fragrances and/or odor neutralizers include one or more antioxidants. Antioxidants used in embodiments of the invention may be, for example, tertiary butylhydroquinone, n-octadecyl 3,5-di-tert-butyl-4-hydroxyhydrocinnamate, butylated hydroxyanisole, phenol bisphosphite, butylated hydroxytoluene, and phosphite compounds. An effective amount of antioxidant in the instant composition is 0.015% to 2.5% by weight of the EVA or other polymer, preferably 0.1 to 0.75% by weight and most preferably 0.2 to 0.5% by weight. In preferred embodiments of the invention, high concentrations of antioxidants are mixed with fragrance prior to addition of the fragrance/antioxidant mixture to any other components of the mixture.

Still further embodiments of the invention contemplate inclusion of the fragrance and/or odor neutralizer in a diluent prior to incorporation into a polymeric carrier. A diluent is organic, for example: triethyl citrate; di-isopropropyl adipate; di-octyl adipate; isopropyl myristate; isopropyl palmitate; butyl stearate; benzyl alcohol; benzyl benzoate; and diethyl pthalate. The quantity of diluent preferred is the quantity necessary for dissolving the fragrance or the antioxidant.

In one preferred embodiment, a selected fragrance and/or an odor neutralizer (with or without the other additives reported above) is embedded in and/or adsorbed on the polymer. Further information regarding creation of a fragrance/antioxidant/diluent mixture may be found in U.S. Pat. No. 7,220,288, which is incorporated by reference as if fully rewritten herein.

Various other additives such as color additives may be added in different embodiments depending on desired characteristics of a particular fragrance device.

Plasticizers may also be added to polymeric materials that are used in embodiments of the invention. These may include, for example, diethyl phthalate and tri-acetic acid ester of glycerin.

### EXAMPLES

The following examples are given to help those skilled in the art appreciate the invention. They should not be construed to limit the scope of the claims.

### Example 1

Example 1 describes the construction of a polymer support containing a fragrance. An amount of fragrance that is 33.4% by weight of the anticipated final formula amount is weighed and loaded into a vessel. Into the fragrance is added antioxidant and hindered amine at 0.7% and 0.7% by weight of the anticipated final formula amount, respectively. An amount of 0.2% of a color solution is added. This is mixed until dissolution.

The fragrance solution is transferred to a 5 gallon drum. EVA in the amount of 65% by weight of the anticipated final formula amount is added as solid beads. The drum is sealed and rotated until the fragrance mixture is completely absorbed into the EVA.

The beads are loaded into an injection molding machine. It is preferred that prior to injection molding the beads be no larger than 325 mesh. A fragrance support is created in the desired shape through the injection molding process. The finished fragrance support may be packaged for sale as a refill item or may be placed within a housing.

Once the polymer support has been created, it is inserted into a housing. The housing is, for example, of a clamshell design. Opposite sides of the housing may be held by an interference fit, by an adhesive, by melting, or by other methods as appreciated by those in the art.

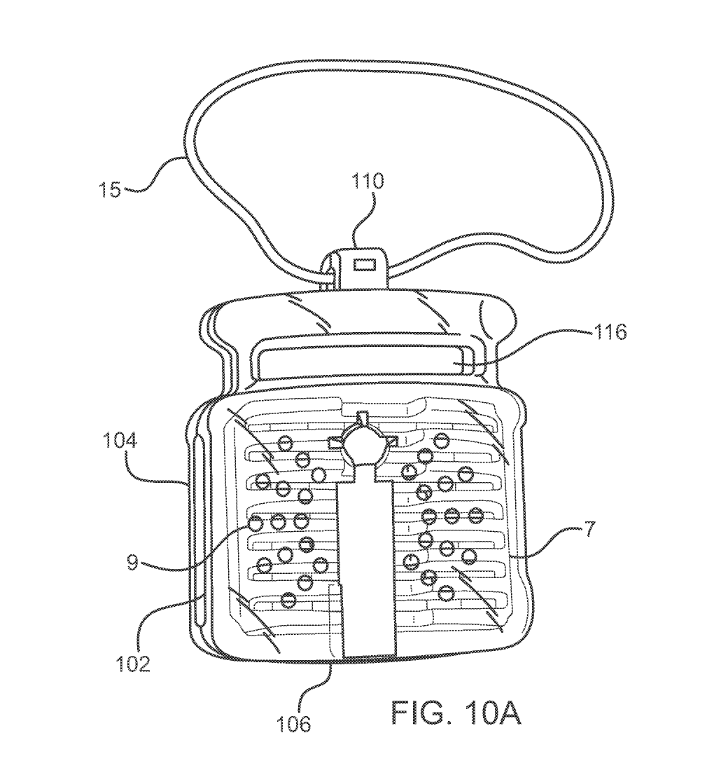

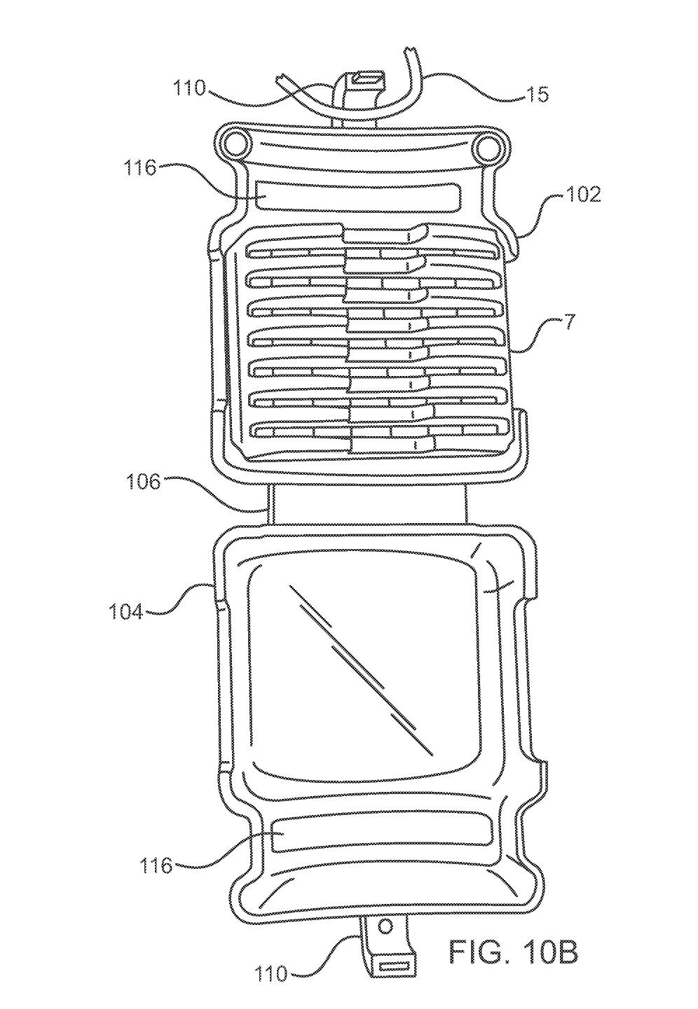

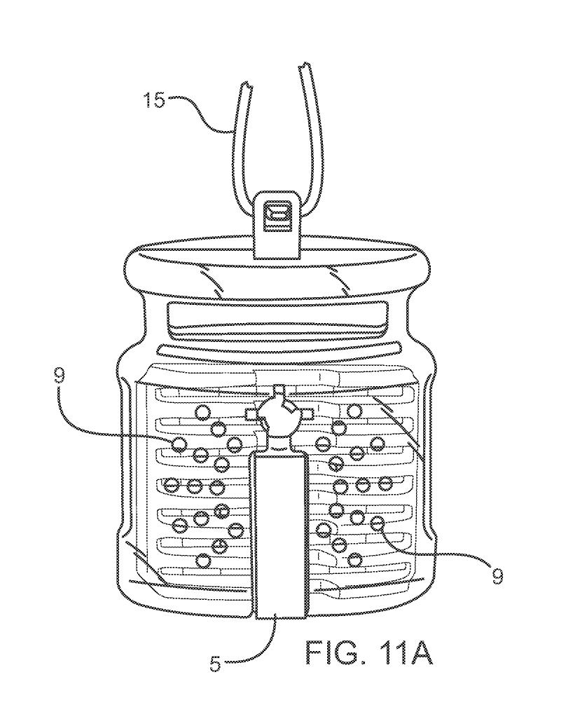

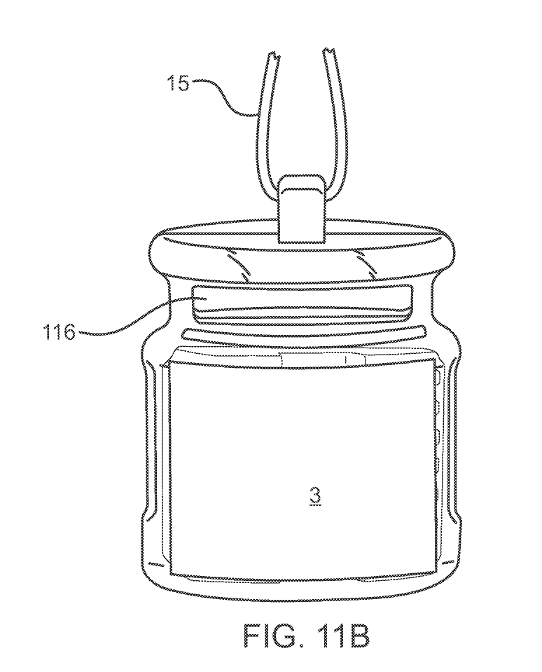

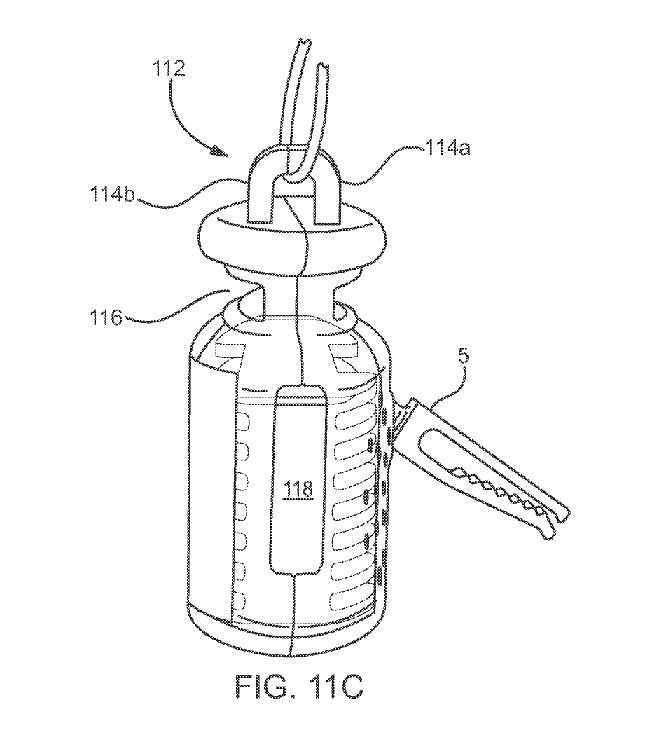

Referring to FIGS. 10 and 11, embodiments of the invention with a housing 1 having hingedly connected compartments 102, 104 are disclosed. In an alternative embodiment, the housing 1 may include a first compartment 102 and a second compartment 104 attached via a hinged engagement 106 so that the first compartment 102 can rotate about an axis defined by the hinged engagement 106 to facilitate enveloping a carrier/support 7 within at least one cavity of a compartment 102, 104. The housing 1 may be fabricated from a lightweight material such as plastic, a polymer, metal, etc. The housing 1 may be opaque, translucent, or transparent to conceal, obscure, or reveal the carrier/support 7 placed therein.

The hinged engagement 106 can include a flexible member connecting a portion of the first compartment 102 to a portion of the second compartment 104. Other hinged engagements 106 may include, but are not limited to, a curved extension member extending from the first compartment 102 engaged with a slotted extension member extending from the second compartment 104, a pin and hinge assembly, a pin and hub assembly, etc. The portions connected by the hinged engagement 106 may be the bottom portions of each compartment 102, 104; however, any portion of the compartments 102, 104 that can be hingedly connected to facilitate selective envelopment of the carrier/support 7 can be utilized.

The first compartment 102 may be structured to form a general rectangular shape with a first cavity formed as a recess within an interior surface thereof. The second compartment 104 may be similarly structured. While the compartments 102, 104 may be described and illustrated as having rectangular shapes, they may have other shapes, such as triangular, spherical, hexagonal, etc. It is understood that only one compartment 102, 104 or both compartments 102, 104 may have a cavity formed within their interior surfaces. The cavity(ies) can form a hollow interior to the housing 1 when the first compartment 102 is rotated about the hinged engagement 106 to abut against the second compartment and generate a closed position At least one cavity may exhibit a shape to complement that of the carrier/support 7, but it is not necessary to match that of the carrier/support 7. At least one cavity of a compartment 102, 104 may be configured to receive at least half the thickness of the carrier/support 7 when placed therein. Once placed inside a cavity(ies), a compartment 102, 104 may be rotated about the axis defined by the hinged engagement to abut against the opposing compartment 104, 102. As the first compartment 102 abuts the second compartment 104, the housing 1 may envelop the carrier/support 7 placed therein, wherein the structure of the cavity(ies) can be such that they receive and envelop the carrier/support 7.

A portion of the housing 1 may be provided with a fastening mechanism 110 to temporarily retain the housing 1 in the closed position, where the closed position may be defined by the compartments 102, 104 abutting each other, as described above. The fastening mechanism 110 can be positioned on any portion of the housing 1 to facilitate retaining the housing 1 in the closed position. In an exemplary embodiment, the fastening mechanism 110 can be located at a top portion of the housing 1 and the hinged engagement 106 can be located at the bottom portion of the housing 1. The fastening mechanism 110 can include a first fastening component located on the first compartment 102 and a second fastening component located on the second compartment, where each are configured to align with and engage each other when the housing 1 is in the closed position. The fastening mechanism 110 can be structured to operate by a protrusion configured to extend into a receptacle and form an interference/snap fit with each other. Other fastening mechanisms 110 may include, but are not limited to, a mechanical clasp, interference tab engagement, magnetic clasp, etc. The fastening mechanism 110 may enable a user to easily and quickly remove, replace, and replenish carriers/supports 7, as needed.

A top portion of the housing 1 may include a lanyard ring 112 configured to receive the loop 15 (see FIG. 11C). In some embodiments, the lanyard ring 112 is bifurcated so that a first lanyard portion 114a is affixed to the first compartment 102 and a second lanyard portion 114b is affixed to the second compartment 104. As shown in FIG. 10B, the fastening mechanism 110 may be disposed within the portions 114a, 114b and operate by an interference/snap fit of a protrusion configured to extend into a receptacle. For example, the protrusion may disposed on the second portion 114b while the receptacle is formed into the first portion 114a. The housing 1 may be configured such that as the compartments 102, 104 are rotated to form the closed position, the protrusion of the second portion 114b may engage the receptacle of the first portion 114a to fasten the housing 1 in a closed position and to form the lanyard ring 112.

In the alternative, or in addition, the housing 1 may include a bifurcated aperture 116, which may serve as a secondary lanyard ring. The bifurcated aperture 116 may include a first aperture component formed into the first compartment 102 adjacent the first cavity and a second aperture component formed into the second compartment 104 adjacent the second cavity such that the first and second aperture components align to form the bifurcated aperture 116 when the housing 1 is in the closed position. The loop 15 may be routed through the bifurcated aperture 116 after the housing 1 is in the closed position, which may be desirable in situations where routing the loop 15 through the lanyard ring 112 is impracticable and/or generates a tendency to unfasten the fastening mechanism 10 (e.g., the loop is too thick, the fastening mechanism is damaged or worn, etc.). The housing 1 may be configured such that as the compartments 102, 104 are rotated to form the closed position, the first and second aperture components align to form the lanyard ring 112.

At least one of the first and second compartments 102, 104 may include the clip 5 disposed on an external surface thereof, in a similar manner as described above. One skilled in the art, with the benefit of the present disclosure, will appreciate that other attachment devices may be used instead of the clip. These may include an adhesive peg, a hook, etc. Use of the clip 5 may enable removably attaching the housing 1 to an ancillary structure via securement by the clip 5. As described above, the clip 5 can be secured to the compartment 102, 104 by a screw, an interference fit, or by a ball and socket attachment. In addition, a portion of the compartment 102, 104 having the clip 5 may include a recess 30 formed into the external surface thereof. The clip 5 may be attached to the housing 1 at the location of the recess 30 or at a location adjacent the recess 30.

With the ball and socket attachment embodiment, the recess 30 may be structured to receive the clip 5 by rotating the clip 5 about the pivot defined by the ball/socket and into the recess 30 for a stowed position. Rotating the clip 5 from within the recess 30 can be done to place the clip 5 in a deployed position, which may enable removably attaching the housing 1 to an ancillary structure via securement by the clip 5. The recess 30 may be configured to form an interference fit with the clip 5 when rotated into the stowed position to retain the clip 5 in place. In addition, the ball and socket pivot assembly may be structured such that the ball is formed on a distal end of the clip 5 and the socket is formed into the surface of the compartment 102, 104. However, the socket can be on the clip 5 and the ball on the compartment 102, 104. The ball and socket assembly may be configured to enable removable engagement of the ball with the socket to facilitate removabe securement of the clip 5 to the housing 1. Alternatively, the clip 5 may be permanently secured to the housing 1.

The clip 5 may be removably attached without a pivoting engagement. For instance, the clip 5 may be attached via an interference with between a protrusion of the clip and a receptacle of the compartment 102, 104. In some embodiments, the clip 5 can be completely removed from the housing 1 and secured within the recess 30 to be placed in a stowed position. In such a stowed position, the clip 5 may lay within the recess 30 so as to be approximately flush with, or even counter-sunk within, the outer surface of the compartment 102, 104, held in place by the interference fit between the clip 5 and the recess 30. The clip may then be removed from the recess 30 and placed into a deployed position by inserting the protrusion of the clip 5 into the receptacle of the compartment 102, 104.

Similar to the embodiment described above, the label 3 may be positioned on an inner and/or outer surface of at least one compartment 102, 104. Attachment of the label 3 may be achieved via an adhesive. The label 3 can be configured to cover a face of a compartment 102, 104 and conceal the carrier/support 7 within the housing 1.

As described above, at least one fragrance vent 9 can be included in any compartment 102, 104. The fragrance vents 9 may be an aperture or other opening formed into a compartment 102, 104 so as to penetrate through the compartment 102, 104 and enable fluid communication between the hollow interior of the housing 1 and an exterior environment. While it is shown for the fragrance vent 9 to be positioned on the second compartment 104 in FIGS. 11A and 11B, the fragrance vent 9 can be placed on any one or both compartments 102, 104.

At least one bifurcated slot 118 may be formed into the housing 1. The bifurcated slot 118 may be used to facilitate release of fragrance from the hollow interior of the housing 1 from side portions of the housing 1 by enabling fluid communication between the hollow interior of the housing 1 and the exterior environment. The bifurcated slot 118 may include a first slot component formed into the first compartment 102 adjacent the first cavity and a second slot component formed into the second compartment 104 adjacent the second cavity such that the first and second slot components align to form the bifurcated aperture 116 when the housing 1 is in the closed position.

I claim:

1. A fragrance device comprising:
a housing comprising a hollow interior, at least one vent placing the interior in communication with an external environment, and a recess in an external wall of the housing; and,
an attachment device, said attachment device capable of being disposed within the recess and capable of being removed from the recess and secured to the housing.

2. The fragrance device of claim 1, further comprising a fragrance support within the hollow interior, said support including at least one of a fragrance, an insect repellent, and an odor neutralizer, wherein said fragrance support comprises at least one of a polymer fragrance support, a gellant fragrance support, and a non-polymer fragrance support.

3. The fragrance device of claim 1, wherein the attachment device, when disposed within the recess, is covered by at least one member of the group consisting of a decal and a hinged cover.

4. The fragrance device of claim 1, wherein the attachment device is removably secured to the housing.

5. The fragrance device of claim 1, wherein the attachment device is permanently secured to the housing.

6. The fragrance device of claim 1, wherein the attachment device is selected from the group consisting of a clip, an adhesive peg, and a hook.

7. The fragrance device of claim 1, wherein the attachment device is secured to the housing by a member of the group consisting of a screw, an interference fit, an adhesive, and a ball and socket.

8. The fragrance device of claim 1, wherein the polymer fragrance support is ethyl vinyl acetate.

9. The fragrance device of claim 1, wherein the fragrance includes at least one member of the group consisting of benzaldehydes, phenols, cinnamic aldehydes and esters, octadienes, dienes, cyclohexadienes, and terpenes.

10. A fragrance device comprising:
a housing comprising a first compartment and a second compartment attached via a hinged engagement, wherein said first compartment has a first cavity and said second compartment has a second cavity forming a hollow interior of the housing when said first compartment is rotated about said hinged engagement to abut against said second compartment and generate a closed position;
at least one fragrance vent placing said hollow interior in fluid communication with an external environment;
a fragrance support comprising at least one of a polymer fragrance support, a gellant fragrance support, and a non-polymer fragrance support configured to fit within said hollow interior, said fragrance support including at least one of a fragrance, an insect repellent, and an odor neutralizer;
a recess formed into an exterior surface of at least one of said first and second compartment; and,
an attachment device comprising clip pivotally attached to an external surface of at least one of the first and second compartment, said clip capable of being rotated and received by said recess to form a stowed position and capable of being rotated from said recess to form a deployed position, wherein said clip is configure to removably secure said housing to an ancillary structure;
wherein said first compartment and said second compartment is rotatable about said hinged engagement to facilitate envelopment of said fragrance support within said housing.

11. The fragrance device of claim 10, further comprising a fastening mechanism to temporarily retain said housing in said closed position.

12. The fragrance device of claim 10, further comprising a lanyard ring disposed on a portion of said housing configured to receive a loop.

13. The fragrance device of claim 10, further comprising a bifurcated aperture including a first aperture component formed into the said compartment adjacent said first cavity and a second aperture component formed into said second compartment adjacent the second cavity such that said first and second aperture components align to form said bifurcated aperture when said housing is in said closed position.

14. The fragrance device of claim 10, further comprising at least one bifurcated slot including a first slot component formed into said first compartment adjacent said first cavity and a second slot component formed into said second compartment adjacent said second cavity such that said first and second slot components align to form said bifurcated aperture when said housing is in said closed position.

15. The fragrance device of claim 10, wherein said attachment device is removably secured to said housing.

16. The fragrance device of claim 10, wherein said attachment device is permanently secured to said housing.

17. The fragrance device of claim 10, wherein said attachment device comprises at least one of an adhesive peg and a hook.

18. The fragrance device of claim 10, wherein said attachment device is secured to said housing by a member of the group consisting of a screw, an interference fit, an adhesive, and a ball and socket.

19. The fragrance device of claim 10, wherein said polymer fragrance support is ethyl vinyl acetate.

20. The fragrance device of claim 10, wherein said fragrance includes at least one member of the group consisting of benzaldehydes, phenols, cinnamic aldehydes and esters, octadienes, dienes, cyclohexadienes, and terpenes.
